(19) United States
(12) Plant Patent Application Publication
(10) Pub. No.: US 2014/0283232 P1
(43) Pub. Date: Sep. 18, 2014

(54) Blueberry plant named 'C04-014'

(50) ;

(71) Applicants: Florida Foundation Seed Producers, Inc., Marianna, FL (US); CostaExchange Pty Ltd., Corindi (AU)

(72) Inventors: Gary Wright, Corindi Beach (AU); Paul Lyrene, Micanopy, FL (US)

(21) Appl. No.: 13/999,604

(22) Filed: Mar. 12, 2014

Related U.S. Application Data
(60) Provisional application No. 61/851,672, filed on Mar. 12, 2013.

(51) Int. Cl. A01H 5/00

(52) U.S. Cl. PLT/157

(57) ABSTRACT

A new and distinct cultivar of blueberry (Vaccinium corymbosum hybrid) plant named ‘C04-014,’ characterized by its semi-upright plant shape and strong plant growth vigor, late timing of fruiting, medium to large fruit size, firm fruit suited to handling, good fruit flavor. This combination results in higher quality fruit with a later availability than other varieties.

### LATIN NAME OF THE FAMILY, GENUS, AND SPECIES

Family—Ericaceae

Genus—Vaccinium

Species—corymbosum hybrid

### VARIETY DENOMINATION

The new blueberry plant claimed is of the variety denominated ‘C04-014.’

### BACKGROUND OF THE INVENTION

The present invention relates to a new and distinct perennial variety of Vaccinium corymbosum hybrid (blueberry), which has been given the variety denomination of ‘C04-014.’ The new variety ‘C04-014’ shows distinctive traits such as late season, strong plant vigor, medium to large fruit of good flavor, firm fruit. The new variety ‘C04-014’ is intended for use as fresh fruit for shipping, customer pick and processing markets and as a home garden plant.

The new blueberry cultivar is a selection resulting from seedlings produced in a controlled breeding programme of Vaccinium varieties in Florida, USA in 2002 from a cross of the blueberry variety known as ‘Star’ (seed parent) (unpatented) and the blueberry variety known as ‘C96-97’ (pollen parent) (unpatented). The seed from the cross was sown and grown in Corindi Beach, New South Wales, Australia. The new cultivar was discovered and selected in 2004 as a single plant within a population of seedlings resulted from the controlled cross, in an experimental block in the field at Corindi Beach, New South Wales, Australia, and has since been named ‘C04-014.’ Selection criteria were a combination of late season, strong plant vigor, medium to large fruit of good flavor and firm fruit. The new variety was subsequently evaluated for a number of years at the commercial farm at Corindi Beach, New South Wales, Australia.

Asexual reproduction of the new variety ‘C04-014’ by softwood cutting propagation since 2004 at Corindi Beach, New South Wales, Australia has demonstrated that the new variety reproduces true to type with all of the characteristics, as herein described, firmly fixed and retained through successive generations of such asexual propagation, with the clones phenotypically identical to the original plant.

The seed parent ‘Star’ is characterized by an upright growth habit and early to medium timing of ripening or fruiting. The pollen parent ‘C96-97’ is characterized by a weak to medium plant growth vigor and firm fruit. The new variety differs from the seed parent ‘Star’ in that ‘C04-014’ has later fruiting season. The new variety differs from the pollen parent ‘C96-97’ in that ‘C04-014’ has stronger plant vigor. The new variety ‘C04-014’ has maintained its distinguished characteristics throughout successive asexual propagation.

### SUMMARY OF THE INVENTION

The following characteristics of the new variety have been repeatedly observed and can be used to distinguish ‘C04-014’ as a new and distinct variety of Vaccinium corymbosum hybrid:

1. Late fruiting season

2. Strong plant vigor

3. Semi-upright growth habit

4. Long leaves

5. Medium-large fruit

6. Firm fruit

Plants of the new variety ‘C04-014’ differ from the seed parent ‘Star’ in that ‘C04-014’ has later fruiting season. The new variety differs from the pollen parent ‘C96-97’ in that ‘C04-014’ has stronger plant vigor. The new blueberry variety ‘C04-014’ has maintained its distinguished characteristics throughout successive asexual propagation.

### BRIEF DESCRIPTION OF THE DRAWINGS

The accompanying photographic illustration shows typical specimens in full color of the foliage and fruit of the new variety ‘C04-014.’ The colors are as nearly true as is reasonably possible in a color representation of this type.

FIG. 1 is a photograph of the new variety ‘C04-014,’ demonstrating the plant's semi-uptight habit.

FIG. 2 is a photograph of the new variety ‘C04-014,’ showing parts of the plant in comparison with other varieties.

FIG. 3 is a photograph of the flowers of the new variety ‘C04-014.’

FIG. 4 is a photograph of the summer foliage of the new variety ‘C04-014.’

The colors in the photographs are as close as possible with the photographic and printing technology utilized. The color values cited in the detailed botanical description accurately describe the colors of the new blueberry.

### DETAILED BOTANICAL DESCRIPTION

The following detailed description sets forth the distinctive characteristics of ‘C04-014.’ The data which defines these characteristics was collected from asexual reproductions of the original selection. Dimensions, sizes, colors, and other characteristics are approximations and averages set forth as accurately as possible. The plant history was taken on plants approximately 4 years of age, and the descriptions relate to plants grown in the field in Corindi Beach, New South Wales, Australia. Descriptions of fruit characteristics were made on fruit grown in Corindi Beach, New South Wales, Australia. Color designations are from RHS Chart—edition 2007.
Classification:
   Family.—Ericaceae.
   Genus.—Vaccinium.
   Species.—corymbosum hybrid.
   Common Name.—blueberry.
Parentage:
   Seed parent.—‘Star’ (unpatented).
   Pollen parent.—‘C96-97’ (unpatented).
Market class: Suitable for the Commercial, Hand Harvest Fresh, Processed, and Home Garden Markets
Plant:
   Parentage.—‘Star’×‘C96-97’.
   Growth habit.—Semi-upright.
   Growth.—Vigorous.
   Productivity.—Medium to high.
   Chilling requirement.—Low to medium chill.
   Leafing.—Moderate.
Foliage:
   Time of beginning of leaf bud burst.—Around the first week of September, similar to ‘Star’.
   Leaf color (top side).—Similar to green group 137A and the vein color similar to yellow-green group 145B.
   Leaf shape.—Elliptic.
   Leaf margins.—Minor serration.
   Leaf length.—Average 88.2 mm (range 76-95 mm).
   Leaf width.—Average 37 mm (range 28-44 mm).
   Leaf nectarines.—Absent.
   Pubescence of upper side.—Absent.
   Petioles.—a. Length: Average 6.4 mm (4.6-8.9 mm).
Flowers:
   Time of beginning of flowering.—Medium to late, around middle August.
   Time of 50% anthesis.—15^{th} August.
Fruit:
   Time of fruit ripening.—Late season.
   Time of 50% maturity.—Average 6^{th} November.
   Unripe fruit color.—Similar to yellow-green group 144B.
   Ripe berry color.—Similar to Blue group 103A.
   Berry weight.—3 g.
   Sweetness when ripe.—Medium to high (average 15 brix).
   Firmness when ripe.—High.
   Acidity when ripe.—Medium to high.
   Uses.—Hand harvest fruit.
Seed:
   Seed abundance in fruit.—Low, similar to ‘Star’.
   Seed length.—Average 1.9 mm (range 1.6-2.2 mm).

1. A new and distinct variety of blueberry plant named ‘C04-014,’ substantially as illustrated and described herein.
